(19) United States
(12) Patent Application Publication
(10) Pub. No.: US 2019/0361427 A1
(43) Pub. Date: Nov. 28, 2019

(54) SYSTEM AND METHOD FOR MECHANICAL FASTENING DESIGNS

(71) Applicant: Mediprint, LLC, Goshen, NY (US)

(72) Inventor: Brent Chanin, Goshen, NY (US)

(73) Assignee: Mediprint, LLC, Chester, NY (US)

(21) Appl. No.: 16/515,908

(22) Filed: Jul. 18, 2019

Related U.S. Application Data
(63) Continuation of application No. 16/372,947, filed on Apr. 2, 2019.
Application No. 16/372,947 is a continuation of application No. 16/373,114, filed on Apr. 2, 2019.
(60) Provisional application No. 62/651,530, filed on Apr. 2, 2018.
(60) Provisional application No. 62/651,545, filed on Apr. 2, 2018.
(60) Provisional application No. 62/651,530, filed on Apr. 2, 2018.
(60) Provisional application No. 62/651,545, filed on Apr. 2, 2018.
(60) Provisional application No. 62/651,562, filed on Apr. 2, 2018.
(60) Provisional application No. 62/699,827, filed on Jul. 18, 2018.

(51) Int. Cl. G05B 19/4099; G16H 30/20; G06F 3/12; B33Y 50/00

(52) U.S. Cl. CPC G05B 19/4099; G16H 30/20; G06F 3/1297; B33Y 50/00; G06F 3/1286; G06F 3/1232; G05B 2219/49007; G05B 2219/35134; G06F 3/1206

(57) ABSTRACT

The present disclosure describes systems and methods to create three-dimensional tangible models which may be taken apart and reassembled with ease and accuracy. The three-dimensional tangible models may be segmented, layered, or otherwise able to be disassembled and reassembled again, so one is able to present views of an interior or otherwise obscured portion of a tangible object. In some examples, a tangible segmented model corresponds to a patient-specific body part.

### CROSS REFERENCES TO RELATED APPLICATIONS

This application claims priority to U.S. Provisional Patent Application Ser. No. 62/699,827, entitled “SYSTEM AND METHOD FOR MECHANICAL FASTENING DESIGN”, filed on Jul. 18, 2018 and as a continuation to U.S. Non-Provisional patent application Ser. No. 16/372,947 tilted “APPARATUS AND METHODS FOR CREATION OF A PATIENT-SPECIFIC ANATOMICAL MODEL”, filed on Apr. 2, 2019 and as a continuation to U.S. Non-Provisional patent application Ser. No. 16/373,114 entitled “APPARATUS AND METHODS FOR CREATION OF A PATIENT-SPECIFIC BODY PART OR MEDICAL INSERT”, filed on Apr. 2, 2019 each of which are relied upon and incorporated herein by this reference.

### FIELD OF THE DISCLOSURE

The present disclosure relates to systems and methods to create tangible three-dimensional models which may be taken apart and reassembled with ease and accuracy.

### BACKGROUND OF THE DISCLOSURE

Complex three-dimensional models have many applications in medicine, teaching, and science. One benefit to three-dimensional models is a display of internal structures. However, three-dimensional models may be realized in physical space in numerous manners. In various examples, the internal structure of the physically rendered model may become impossible or difficult to visualize due to being imbedded behind externally displayed features. In some examples, the physical three-dimensional models may be composed of materials that transmit light and thus allow for internal structure to be visualized if that structure is able to be understood from the nature of the transmitted light. For example, color variation may be used to display shapes and contours of the internal structure. Even so, the visualization of the internal structure may be difficult, and distortions of the visualized structure occur. For this reason, it may be common to create cross sections of the physical model and display the structure on the surface of the cross section.

Cross-sectioned pieces may have complicated shapes, and although it is typically straightforward to separate pieces that have been cut in cross section, it may be very difficult to reorient these pieces in their correct configuration. It would be very helpful if improved model generation techniques were developed which allowed for easy separation as well as accurate and easy reassembly. Additional improvements accrue from techniques which enable nimbleness, which are cross-compatible, and which can be operated single-handedly. Other desirable attributes may include solutions which have joints and are articulated, attractive, sturdy, or intuitive.

A novel class of such three-dimensional models includes medical models which have many uses in the medical industry. Non-specific models of human physiology (i.e., models that are not limited to a specific person) are useful as learning tools in contexts such as anatomy class in medical school, medical instructions that affect a group of patients (e.g., for conditions such as pregnancy, insertion of an intra-uterine device, disorders such as enlarged hearts, etc.), cardio-pulmonary resuscitation (CPR) training, and so forth.

In contrast, a patient-specific medical model is a medical model of at least a portion of the anatomy (internal or external) of one specific patient, at one point in time. The patient-specific medical model may be useful to a medical practitioner to understand a patient's medical state, to plan a medical procedure for the patient, and/or to perform the medical procedure for the patient. A modality of the patient-specific medical model may refer to the technology used to obtain the patient-specific medical model.

One technology of the background art to obtain a patient-specific medical model is by computed tomography (CT) scan, also referred to as computed axial tomography or computer aided tomography (CAT) scan. CT and CAT scans make use of computer-processed combinations of many X-ray measurements taken from different angles to produce cross-sectional (tomographic) images (virtual “slices”) of specific areas of a patient or other scanned object, allowing a medical professional to see inside the patient without cutting open the patient.

Another technology of the background art to obtain a patient-specific medical model is by magnetic resonance imaging (MRI). An MRI scanner uses strong magnetic fields, electric field gradients, and radio waves to generate images of the organs in the body. The images generally are of a planar slice through the patient's body. MRI does not involve X-rays and the use of ionizing radiation, in contrast to CT or CAT scans. Compared with CT scans, MRI scans typically take longer and are louder, and they usually need the patient to enter a narrow, confining tube. In addition, patients with some medical implants or other non-removable metal inside the body may be unable to undergo an MRI examination safely.

Data from a CT scan or MRI scan is ordinarily stored in accordance with NEMA PS3/ISO 12052, Digital Imaging and Communications in Medicine (DICOM) Standard, National Electrical Manufacturers Association, Rosslyn, Va., USA (available free at http://medical.nema.org/). The DICOM standard covers both the formats to be used for storage of digital medical images and related digital data, and the protocols to be adopted to implement several communication services which are useful in the medical imaging workflow. DICOM facilitates cross-vendor interoperability among devices and information systems dealing with digital medical images.

Having an accurately and timely patient-specific medical model is extremely important to the successful performance of critical procedures such as surgeries, or treatment of delicate tissue such the eye or nerve tissue. Surgeries benefitting from patient-specific models may include cardiac surgery, neurosurgery, oncology surgery, ophthalmologic surgery, orthopedic surgery, plastic surgery, and so forth. A preferred physical resolution (e.g., slice thickness) in the background art of the patient-specific medical model may depend upon the type of surgery and the modality used to obtain the model data, and generally ranges from less than about 2.0 mm to less than about 0.7 mm of precision.

In connection with the critical procedures, a replacement body part may be needed, e.g., a prosthetic or a replacement for an internal body part. For example, the replacement for an internal body part may be replacement for a joint (e.g., hip, knee, elbow, etc.), a bone (e.g., a vertebrae), a cartilage (e.g., ear, nose, etc.), reconstructive surgery (e.g., to correct a birth defect, to correct a traumatic injury, etc.), a blood vessel to replace a clogged vessel, and so forth. The replacement body part may be used for other purposes, instead of or in addition to being used as a replacement, such as being used as a physical model of a body part for illustrative or learning purposes, etc.

All of these different medical models share the general needs as mentioned initially for improvements that make it easy to demonstrate internal structure and disassemble and reassembly them for various purposes.

### SUMMARY OF THE DISCLOSURE

Accordingly, apparatus and methods to present sectioned physical models in a manner that allows for straightforward disassembly and reassembly to allow for inspection of internal structure on cross sectioned pieces are presented. By creating a tangible model which is segmented, layered, or otherwise able to be disassembled and reassembled again, one is able to present views of the interior or otherwise obscured portions of an object. In some examples, the segmented model corresponds to a patient-specific body part. The replacement body part is created from a model that is produced in vivo, non-invasively, and with sufficient accuracy and detail to be used to guide later surgery.

Means to separate the models into parts may include slicing a model into slabs and installing magnets within pre-designed cavities that help confer correct orientation. In some other examples, the model may be sliced into slabs which align with a suspended length of mating contacts along an axle. Models may attach via fasteners similar to those described above, however they may be oriented in the orthogonal direction from the slice plane.

Embodiments in accordance with the present disclosure provide for a system and method to create a patient-specific replacement body part which may then be displayed in manners that allow for easy disassembly and reassembly. Imaging scans are acquired by modalities such as magnetic resonance imaging (MRI) or computed tomography (CT), and are stored as image data in standard DICOM format in order to promote data portability and interoperability. The data may be further processed to a format compatible with a fabrication apparatus such as a 3-D printer or other automated maker to directly create medical models or medical replacement parts from the imaging scans.

In the following sections, detailed descriptions of examples and methods of the disclosure will be given. The description of both preferred and alternative examples though through are exemplary only, and it is understood that to those skilled in the art that variations, modifications, and alterations may be apparent. It is therefore to be understood that the examples do not limit the broadness of the aspects of the underlying disclosure as defined by the claims.

### BRIEF DESCRIPTION OF THE DRAWINGS

The accompanying drawings, that are incorporated in and constitute a part of this specification, illustrate several embodiments of the disclosure and, together with the description, serve to explain the principles of the disclosure:

FIG. 1 illustrates a system to create a patient-specific medical model, in accordance with an embodiment of the present invention;

FIG. 2 illustrates a method to create a patient-specific medical model, in accordance with an embodiment of the present invention;

FIG. 3 illustrates an embodiment at a low level of abstraction of a system to design a patient-specific medical model, in accordance with an embodiment of the present invention;

FIG. 4 illustrates a method to design a patient-specific medical model, in accordance with an embodiment of the present invention;

FIG. 5 illustrates a method to create a patient-specific medical model, in accordance with an embodiment of the present invention;

FIGS. 6A-6C illustrate different views of a sectioning of a medical model in accordance with an embodiment of the present invention;

FIG. 7A-7C illustrate different views of a sectioning of a medical model in accordance with an embodiment of the present invention; and

FIG. 8 illustrates a pattern of coupling mechanisms that may be included in some embodiments of the present invention.

### DETAILED DESCRIPTION

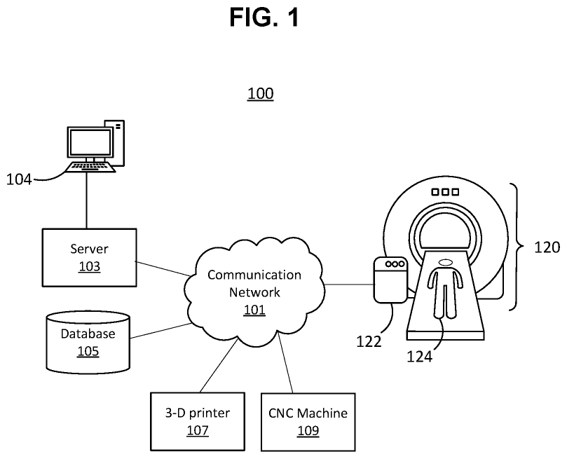

FIG. 1 illustrates a system 100 to design a patient-specific medical model. System 100 includes an MRI machine 120, illustrated as accommodating a patient 124 laying on a gurney and about to undergo MRI examination. MRI machine 120 may include a local control console 122, by which the local operation of MRI machine 120 may be controlled. For example, at local control console 122, an MRI operator (not shown) may control local operation such as movement of the patient gurney to move patient 124 into and out of MRI machine 120, starting and stopping other functions or operating modes of MRI machine 120, etc. Although FIG. 1 illustrates MRI machine 120 as the modality, other modalities such as a CT machine are contemplated as being compatible with system 100.

MRI machine 120 communicatively interfaces with a communication network 101. Communication network 101 may include, e.g., at least a portion of an Ethernet network, the design of which is well known to persons of skill in the art of data networking. The communication interfaces to communication network 101, for MRI machine 120 and other elements of system 100, may be either wired (e.g., CAT-6 cable) or wireless (e.g., Wi-Fi in accordance with standards such as IEEE 802.11). Communication network 101 may represent various configurations such as more than one connected networks (e.g., multiple peer local area networks (LANs)), or networks of differing hierarchies (e.g., one or more LANs coupled to a wide area network (WAN)).

A server 103 may be coupled to communication network 101. Server 103 may be used by a system operator at terminal 104 to control operation of system 100. Terminal 104 may be coupled directly to server 103 as illustrated, or terminal 104 may be coupled through communication network 101 to server 103. Controlling the operation of system 100 may include, e.g., remote configuration of MRI machine 120 specific to the requirements for patient 124 (e.g., configuring the number of scans, field strength, duration of each scan, portion of the body to scan, scan plane, distance between adjacent scans, time between successive scans, data formatting requirements, etc.), and being able to study measurements made by MRI machine 120 and/or computations based upon the measurements. For example, an application program hosted on server 103 may render a motion picture to illustrate time-based or position-based changes in successive scans.

System 100 further may include a database 105, in which MRI scans may be stored for analysis. The data may be stored in a DICOM compatible format. In some embodiments, personally identifiable information (e.g., patient name) may be deleted or not saved with the scan data. In such embodiments, an identification number (e.g., patient ID number) may be saved with the scan data in order to associate the scan data with the correct patient. Database 105 may represent a single database or a distributed database such as a drive array or cloud storage.

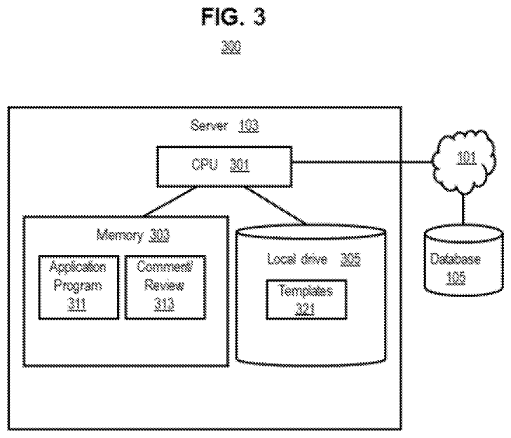

System 100 further may include fabrication apparatus or subsystems to create physical models based upon scan data. For example, FIG. 1 illustrates a 3-D printer 107 and computer numeric control (CNC) machine 109 coupled to communication network 101. Server 103 may include an application program that converts scan data, saved in DICOM format, into a data format and/or file format compatible with a selected fabrication apparatus, such as a standard triangle language (STL) format for 3-D printer 107. Such an application program is described in greater detail with respect to FIG. 3. The fabrication apparatus may create a 3-D model at full size (i.e., life size), or at a selectable percentage of full size for examination purposes (e.g., 50% for a large body part, or 200% for a small body part). A 3-D model to be used as a replacement body part may be ordinarily produced at 100% of full size. Other types of fabrication apparatus also are within the scope of FIG. 1.

As an example of a use-case of system 100, suppose a detailed patient-specific medical model of the liver is needed. For example, the patient may be under consideration for liver surgery, such as to remove cancerous tissue, or a liver transplant (as either a donor or recipient), or to remove damaged portions (e.g., by cirrhosis), or to repair damage such as a torn liver, and so forth. In particular, a multi-layered model of the liver may be needed, e.g., a model of the patient-specific liver with blood vessels, a model of the patient-specific liver with nerves, and so forth. These separate models may be helpful for different types of surgery, or for different phases of a lengthy surgery. The patient-specific model may be augmented with features to improve the usability or the stability of the model, such as by inclusion of physical support features. The physical support features may be rendered or produced in a way to include a feature not seen naturally, e.g., rendering the support features in a physical cross-sectional shape of polygons (such as squares, hexagons, octagons, etc.) in order to differentiate the physical support features from natural features of the patient-specific medical model.

In some embodiments, the physical support features may include features that are usable to couple together different portions of the model. For example, the physical support features may include a detachable attachment feature, such as a hook-and-loop fastener (e.g., Velcro®). The feature may be produced directly by the fabrication apparatus (e.g., 3-D printer 107 prints the hook-and-loop fastener), or the fabrication apparatus may leave a surface or the like where a hook-and-loop fastener produced separately may be applied manually by a model maker.

In some embodiments, the patient-specific medical model may be made from a highly flexible material, e.g., a material whose flexibility mimics the flexibility of (or conversely, the stiffness of) the actual body part that the patient-specific medical model is intended to represent. For example, a patient-specific medical model of blood vessels (either arteries or veins) or the lymph system may be fabricated in a double-wall or double-layer process. A first wall or layer (generically, “first layer”) may be fabricated in the shape of the vessel from a relatively stiffer material. A second wall or layer (generically, “second layer”) then may be fabricated adjacent to the first layer (usually, but not necessarily, outer to the first layer), and being fabricated in a way that is conformal to the first layer. The material used for the second layer may be relatively more flexible than the first layer. After the second layer has been formed, the first layer optionally may be removed (such as by dissolving by a solvent such as acetone that does not significantly affect the second layer), thereby leaving the second layer as a highly flexible patient-specific medical model of the blood vessels.

In some embodiments, the patient-specific medical model made by the fabrication apparatus may have colors selected to provide emphasis to certain features. For example, a 3-D model of a patient-specific heart may be captured at a predetermined point in the heartbeat cycle, and may have clogged portions of various veins and arteries prominently colored if the purpose of the heart model is to plan an open-heart surgery, during which the clogged portions will be treated.

In other embodiments, a specific synthetic body part may be printed from the patient-specific medical model in order to use the synthetic body part as a replacement for a natural body part, e.g., one that is diseased, damaged, congenitally missing or disfigured, has to be replaced with a larger version as a juvenile patient grows, etc. The synthetic body part may be designed using a material that at least mimics observable or experiential characteristics of the natural body part and may surpass the natural body part if appropriate. For example, synthetic body parts to replace cartilage-based body parts such as ears or the nose may be mimic the flexibility of the natural body parts. On the other hand, synthetic body parts to replace bony body parts such as elbows, kneecaps and spine vertebrae may be at least as hard as the natural body part. Synthetic body parts to replace a joint (e.g., knee or hip) may be made from materials having reduced friction during movement, compared to the natural body part.

In some embodiments, the imaging scans used to produce the patient-specific medical model may have been gathered with some disparateness in technical aspects of the imaging scans, e.g., gathered or saved with different data types, formats, intake protocols, varying levels of data validation and of anonymization of protected health information (PHI), and so forth. PHI is known as sensitive health information (e.g., a diagnosis of a psychiatric condition) that is personally identifiable with a specific person, and anonymization of PHI is known as the obscuring of data such that a risk of re-identifying the patient from the protected health information has been reduced below a configurable threshold. Disparities in the technical aspects may have arisen if, e.g., different MRI machines 120 were used (e.g., first at a trauma hospital and later at a reconstruction hospital), or if scans were taken in separate sessions in MRI machine 120, etc. Data validation scripts executing on server 103 may be used to convert the disparate scan data into a common (i.e., shared) format so that the scan data is similar enough to be able to be shared and combined into an improved overall patient-specific medical model.

FIG. 3 illustrates virtualization environments 300 of system 100 at a lower level of abstraction. In some embodiments, memory 303 of server 103 includes an application program 311 in the form of executable instructions that, when executed by central processing unit (CPU) 301, may perform the conversion of data as gathered from MRI machine 120 into models and instructions that can be used by a fabrication apparatus (e.g., 3-D printer 107 and/or computer numeric control (CNC) machine 109, or the like) in order to create a patient-specific medical model. In particular, application program 311 may be used to design a patient-specific customized therapeutic part that can later be fabricated on a fabrication apparatus as a three-dimensional physical model of patient-specific anatomy, for use as a customized therapeutic part, to provide therapy or other medical treatment. The customized therapeutic part also may include portions that are assembled from separate pieces that are not necessarily created on the fabrication apparatus, e.g., pieces that may provide an adhesive, a weave, or a physical trait or material composition that may be difficult to duplicate with fabrication apparatus of the present art, or pieces that may be widely and inexpensively available from generic stock materials (e.g., wire, thread, filament, etc.). The choice of creating a portion of the customized therapeutic part by use of the fabrication apparatus or by assembly from separate pieces may be pre-configured or be controlled by a system operator. The assembly itself may be performed by a skilled human technician.

Server 103 further may include a local drive 305, which in turn may store templates 321. Templates 321 may be used as a library of standard models for generic body parts, i.e., body parts that are not patient specific and not necessarily derived just from data obtained by MRI machine 120. Standard models of generic body parts may be used when a patient-specific model is not needed for all body parts in a model. For example, if a model is needed for repairing knee ligaments, a standard model may be used to represent the knee cap.

In some embodiments, virtualization environments 300 may represent elements accessible over network 101 via remote desktop protocol. In some embodiments, portions 300 may represent an elastic cloud-based computing environment, such as VMware®, Amazon Web Services®, Microsoft Azure®, and so forth.

Memory 303 of server 103 further may include an application program 313 in the form of executable instructions that, when executed by CPU 301, may allow for the review, comment and editing of a model for a patient-specific customized therapeutic part, prior to its fabrication by a fabrication apparatus.

Embodiments in accordance with the present disclosure allow for review and practice of a medical procedure (e.g., a surgery) by a doctor, prior to performing the medical procedure on a live patient. Having a patient-specific customized therapeutic part also may facilitate additional rounds or levels of medical review before a medical procedure is performed on a patient. For example, the patient-specific customized therapeutic part may be used to consult with a more knowledgeable or experienced specialist, or to use for a peer review of a proposed surgery, and so forth. The consultation or review may be performed locally or remotely. The patient-specific customized therapeutic part also may be used to explain a proposed medical procedure to a patient, in order to better explain the procedure to the patient and in order to obtain a more informed patient consent.

The medical procedure also may involve replacing a body part with the patient-specific customized therapeutic part. In some circumstances, when a patient-specific customized therapeutic part is intended to be used for multiple purposes (e.g., to explain a procedure, and to use as a replacement for the body part), separate models may be fabricated. For example, for the purpose of explaining a procedure, a patient-specific customized therapeutic part may emphasize or make more prominent (e.g., by use of a bold color) any unusual anatomy or anticipated problems. On the other hand, a patient-specific customized therapeutic part intended for insertion into a patient may be fabricated from more inert materials, or from a material that is relatively more biologically compatible with human or animal tissue.

In some embodiments, the patient-specific customized therapeutic part may include support features to support other portions of the part. Such support features may be helpful to increase the robustness of otherwise delicate or fragile features. In some embodiments, the support features may be constructed with at least a portion being external to the rest of the patient-specific customized therapeutic part, e.g., provided as wires, threads, filaments, adhesive surfaces, and so forth. In other embodiments, the support features may be constructed with at least a portion being internal to the patient-specific customized therapeutic part, e.g., by use of a relatively stiffer material than the natural counterpart. A stiffer material may be used in concealed locations, e.g., on at least a portion of a concealed surface such as the interior of a vessel, bronchial tube, or other tube-like structures. The concealed surface also may be provided as an interior layer, similar to a fascia, or an outer surface similar to a shell. Such support features may be more useful for patient-specific customized therapeutic parts intended to be used for demonstrative purposes rather than to be used in vivo in the patient.

In some embodiments, the material used for the support surface may be selected to provide a selectable amount of support. For example, the support surface may have varying degrees of rigidity, pliability, softness, flexibility, and so forth. The support surface also may derive at least a portion of its support characteristics from being formed in a rigid lattice-like shape, such as a triangular or hexagonal tessellation.

In some embodiments, the support features may be visibly coded (e.g., by use of a non-natural color) in order to distinguish the support features from the natural portions of the patient-specific customized therapeutic part.

Embodiments in accordance with the present disclosure may include a continuation of anatomical features. For example, the continuation may be user-designated, such as made electronically on an editable MRI scan or on a computer model generated based upon normalized data from multiple MRI scans. Examples of such continuations may include blood vessels, nerves or other anatomical features, or proposed artificial items (e.g., stent, pacemaker, cochlear implant, brain stimulus probe, etc.) as they would be included in and around anatomical features. Such features may be AI-designated and color-coded to distinguish the feature from the rest of the patient-specific customized therapeutic part. Such features also may have a contiguous material and/or surface quality. As with support features described above, the continuation may be fabricated with a selectable amount of tactile characteristics such as varying degrees of rigidity, pliability, softness, flexibility, and so forth. The continuation also may derive at least a portion of its tactile characteristics from being formed in a rigid lattice-like shape, such as a triangular or hexagonal tessellation.

Embodiments in accordance with the present disclosure also may facilitate user control of a three-dimensional model used by the fabrication apparatus. For example, embodiments may allow a user to select a portion of a scan or a model, for generation as a physical representation of the patient-specific customized therapeutic part, based upon normalized data from multiple MRI scans.

Embodiments may allow a user to print only selected portions of the patient-specific customized therapeutic part. For example, a user may be allowed to pan, rotate, zoom, etc. selected portions of the patient-specific customized therapeutic part in order to better illustrate a portion of greater interest. Some embodiments may fabricate outer layers of the patient-specific customized therapeutic part in a transparent or translucent material, in order to make at least partially visible an interior structure of interest. Some embodiments may fabricate portions of the patient-specific customized therapeutic part in special colors. For example, a cancerous organ may be rendered with the cancerous portion of the organ rendered in a different color to visually highlight it. In some embodiments, the patient-specific customized therapeutic part may omit section, portions, features or the like in order to better illustrate features of interest. For example, a patient-specific customized therapeutic part fabricated as a bone in order to illustrate a bone marrow transplant procedure may be fabricated in cutaway form, such that the marrow interior of the bone is visible.

In some embodiments, a patient-specific portion of the body may be designed that is not limited to a single organ. For example, embodiments may include all features in a given area of the body, or multiple specific organs (e.g., related organs like the liver, gall bladder, and pancreas) bones with attached ligaments, etc. In some embodiments, features of less interest may be omitted in order to concentrate attention on the features of greater interest.

In some embodiments, aggregated scan-and-build data may be analyzed to produce an improved patient-specific medical model. For example, artificial intelligence (“AI”) analysis techniques may be used to analyze the injury or disease state of a body part to be modeled or to be replaced, using an AI knowledge base stored in database 105. The AI knowledge base may include, e.g., average or typical scans from other patients to be used for comparison sake.

This may be useful to produce an AI-guided model of therapeutic treatments, e.g., for a specific patient and based upon the patient's specific medical model, produce a prediction of success rate and possible complications from various modeled therapeutic treatments. For example, the AI-guided model may be able to model the alternative outcomes of surgery or no surgery (e.g., for orthopedic injuries), predicted treatment results over time (e.g., how soon after surgery a benefit will be observed, and how long that benefit will last after the surgery), and how the state of a disease or injury will advance over time (e.g., arthritis). The AI-guided model may also simulate a surgery multiple times to compute the risk of a complication and hence develop a risk profile. These results from the AI-guided model may be compared to equivalent predictions based on professional judgment from a health care professional (e.g., a physician rendering an opinion within the area of their expertise) in order to improve over time the quality of the AI-guided model and/or the professional judgment.

Embodiments in accordance with the present disclosure facilitate the selection of materials used in 3D models. For example, material characteristics or material features of the materials may be matched to corresponding characteristics or features of the replacement body part. Material characteristics and material features may include hardness, softness, firmness, flexibility, tackiness, slipperiness, smoothness, roughness, friction or resistance to wear (especially for joints), ductility, brittleness, density, color, porosity, tempered, annealed, tensile strength, molecular or microstructure characteristics (e.g., grain, fibers, nanolattice), thermal resistance, hydrophobic or hydrophilic, radiopacity, and so forth. Certain material characteristics may be avoided when possible. For example, usage of a material attracted to a magnet (e.g., a steel rod) may be avoided. Such materials may be problematic in the presence of magnetic fields (e.g., at a metal detector, in an MRI machine, etc.).

Specific anatomical parts, organs, features, etc., may be characterized in terms of body part characteristics, and the body part characteristics may be associated with the closest available material characteristics or material features. Alternatively, the body part characteristics may be associated with at least a threshold level of the corresponding material characteristic or material feature. Alternatively, the body part characteristic further may be associated with a material having preferably a greater amount of the material characteristic or material feature.

For example, bone may be characterized by an amount of stiffness, or an amount of stiffness per unit cross-sectional area. A major weight-bearing bone may such as a tibia, a femur, or a vertebra in the lower back may have an amount of stiffness that is greater than the stiffness of a non-weight-bearing bone such as a collarbone, a rib, a metacarpal or phlanges bone, etc. A replacement bone part may be made from a material having at least as much stiffness as the corresponding natural bone, and preferably a greater amount of stiffness.

In another example, a replacement joint may have an amount of friction that is less than the friction of a corresponding natural joint, and/or a resistance to wear that is greater than the resistance the wear of the corresponding natural joint. For such a body part, a greater amount of a desirable characteristic (e.g., resistance to wear) is better, and a lesser amount of an undesirable characteristic (e.g., friction) may be better.

For some body parts (e.g., an external body part having at least a portion as an external skin surface), it may be preferable to mimic a characteristic of a natural body part as closely as possible by appropriate selection of material rather than to exceed the characteristic of the natural body part. For example, a prosthetic hand (or ear) preferably should have a texture, softness, firmness, flexibility, tackiness, etc., that matches a natural hand (or ear). Consequently, a silicone or similar material may be useful for an external body part. As a counterexample, a prosthetic hand that is too stiff may be perceived as being claw-like.

For some body parts, it may be preferable to select a material color that matches the color of the original body part. For example, it is more important to match a color of a visible body part such as an ear or prosthetic hand, compared to the color for an internal body part such as a bone, an organ, a stent, etc. In some embodiments, a non-natural color may be used for an internal body part in order to easily identify it as an artificial part during any future surgeries. In some embodiments, several different colors may be used for external body parts because skin tone is not one consistent color but a mixture of textures and colors. Skin around the knuckles, for example, may have more red pigment, as do many palms. The palm of the hand is also commonly lighter in color than the back of the hand and the arm.

In some embodiments, a body part model to be used for illustration or teaching purposes, but not for usage as an operational body part, may be fabricated from a material having a different material characteristic or material feature than the corresponding natural body part. For example, a model of an internal organ (e.g., liver, kidney, etc.) may be fabricated with a non-natural color in order to highlight its position within the torso.

In some embodiments, an application program such as application program 311 may be used to help create the patient-specific body part. For example, application program 311 may have access to one or more of a library of material characteristics for various fabrication materials, and a library of normal characteristics for various natural body parts. The one or more libraries may be stored in a persistent memory such as database 105 or local drive 305. Application program 311 may offer to a user a selection of materials to use when fabricating the patient-specific body part. A best match or suggested match may be offered, based upon a comparison of at least one material characteristic with normal or typical characteristics for the body part. In some embodiments, the selection may be modified by specific abnormal characteristics of a patient-specific body part. For example, if a patient-specific model of a liver scarred with cirrhosis or a skin surface scarred with psoriasis is desired, the material selection for fabrication may be modified based upon the condition the model should include. The selection of fabrication materials may be made by a user using familiar user interface GUI controls such as a drop-down selection list. The selection of fabrication materials also may be influenced by the physical size of the model being produced. For example, if a large portion of the body is being produced, or a relatively smaller portion (e.g., an organ) is being produced at a larger-than-life size, then additional physical support features may be needed. Preferably, any additional physical support features will be provided in a way that is not readily visible to a casual observer.

In some embodiments, fabrication of a model may depend upon a user-controlled quality setting. For example, a high-quality setting may produce a model having superior real-ism for as many body part characteristics as possible. Such a model may be slower to produce or may use more expensive materials. In other circumstances, when a faster or less expensive model is sufficient, a model may be produced with a lower quality setting.

In some embodiments, suitable materials for a replacement body part and/or a body part model may include one or more of silicone, plastic, metal, ceramic, and so forth. All materials should include biocompatibility with human tissue. In some embodiments, a replacement body part such as artificial skin may be fabricated at least in part with a culture of human cells or compatible animal cells (e.g., pig cells) grown on a substrate mesh or membrane.

In some embodiments, a body part model may be fabricated from a luminescent material in order to facilitate illuminating at least a portion of the body part model. In some embodiments, luminescent portions of a body part model may be designed in coordination with light-emitting diodes (LEDs) or other lighting sources. For example, LEDs may be either em-bedded within the body part model as internal illumination, or as external illumination of the body part model, or a combination of both lighting locations. In some embodiments, the body part model may be fabricated to be compatible with a handheld light source, e.g., by having sufficient clearance for the movement of a handheld light source.

In some embodiments, when a replacement body part is being fabricated for use by a patient, a choice and selection of materials may depend upon whether the replacement body part is intended for internal usage (e.g., as orthopedic repair) or for external usage (e.g., as orthopedic support). In some embodiments, a replacement body part and a body part model may be fabricated either sequentially or simultaneously (subject to fabricator apparatus availability), from the same patient-specific medical model.

In some embodiments, when a body part model or a replacement body part re-quires a fastener (such as a screw for, e.g., a dental implant) to secure together pieces or to secure the part to a bone or similar rigid part, application program 311 may design the placement and size of the fastener, and/or a place for the fastener to attach (such as a threaded screw hole). The fasteners themselves may be conventionally supplied medical grade fasteners.

In some embodiments, application program 311 may facilitate the selection of FDA-approved parts based upon the patient-specific medical model, provide a list of any additional required parts in order to verify that the necessary parts and components are on hand and available before a surgery. Doing so helps allow for peer review prior to surgery; helps obtain more informed patient consent prior to surgery and may be able to save time during the surgery itself.

Embodiments in accordance with the present disclosure are not limited to the creation of a patient-specific medical model only for use as a replacement body part or for illustration during training or instructions. Embodiments are also useful to create a patient-specific medical model for use as, e.g., prototypes, final use parts, and emergency usage parts (possibly on a temporary basis) in trauma or triage situations. Fabrication apparatus and its usage will comply with applicable law and regulation, and also preferably adhere to best practices, with respect to: equipment operator identification, qualifications, and training; qualifications and certifications such as ISO 9000; maintenance requirements, logs, and schedules; and material chain of custody such as material sourcing, material quality assurance (QA), and material storage.

Manufacturing QA, including monitoring and observation, may be performed with technological apparatus where feasible, such as cameras for video monitoring and recording, or sensors to detect flaws or assure compliance of individual parts with a design. The fabrication apparatus may provide real-time observation capability to a system operator and may undergo routine maintenance such as scheduled maintenance, cleanings, minor repairs as needed, disposal of replaced parts, and maintaining records thereof including at least some of operator or technician identification, title, dates, etc. Maintenance tools, equipment, and methods may include usage of clay tools and brushes; pressure washing; usage of pumps, tanks or buckets; ultrasonic cleaning; sterilization (e.g., by heat, or chemicals such as alcohol or bleach); and so forth. Cleaning and maintenance procedures will maintain sterility, adhere to biohazard standards when and where applicable, and dispose of waste in a manner commensurate with its risk. If necessary, cleaning and maintenance procedures may be performed by guided or imaging tools, such as a remote-controlled cleaning robot. Where feasible and without creating a biohazard, waste materials may be recycled in accordance with prevailing technology, standards, and initiatives.

In some embodiments, a body part model may be fabricated with an aesthetically pleasing appearance (e.g., a glossy finish) or physical texture (e.g., smooth), as long as the aesthetic artifacts (e.g., surface reflectivity or surface micro-texture) are not important to the demonstrative purpose of the body part model. For example, if a body part model of a liver is used only to illustrate the location of the liver in the abdominal cavity and its placement with respect to other internal organs, then the surface reflectivity and surface micro-texture of the liver model is not important. Standard model-making tools and methods may be used to provide the aesthetically pleasing appearance, such as sanding (e.g., grits of 100-5000), polishing and polish compounds, polishing cloths and sponges, etc. Power tools (e.g., oscillatory or rotary saws, sanders, etc.) may be used. Priming (e.g., heat or chemical) and painting (e.g., spray, brush, roller, air-brush, etc.) may be used.

The fabrication apparatus, its usage, its interface to other portions of system 100, and its scheduling may be automated to the extent feasible (e.g., if justified from a cost/benefit analysis) compared to alternative methods such as a manual method.

In some embodiments, post-process validation may include a manual measurement to verify that parts are being fabricated according to design. For example, validation may include parts scanning (e.g., a laser-based 3D measurement), and a computational comparison of the measurement to the design. Machine learning techniques may be used, e.g., if a systematic bias is found in the 3D measurements. Token verification may also be utilized.

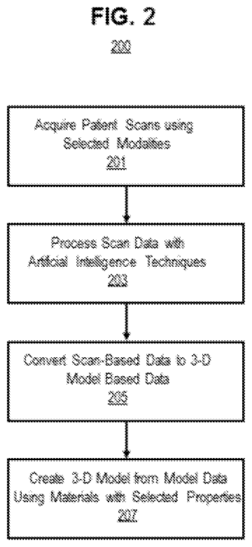

FIG. 2 illustrates a process 200 to design a patient-specific medical model in accordance with an embodiment of the present invention. Process 200 begins at step 201, at which patient-specific scan data is acquired using a selected modality, e.g., by CT scan or by MRI scan. Process 200 continues to step 203, at which artificial intelligence techniques optionally may be applied to the scan data in order to improve the data quality, and to analyze the injury or disease state of body part to be modeled or to be replaced.

Next, process 200 may transition to step 205, at which the scan-based data of previous steps may be converted into a data format usable by a fabrication apparatus or subsystem, such as 3-D printer 107 and CNC machine 109, to design physical models based upon scan data. For example, languages, models and data formats used by a fabrication apparatus may include one or more of G-code, STereoLithography (STL), AMF, ReplicatorG, skineforge, and Cura.

Next, process 200 may transition to step 207, at which a 3-D model may be designed from the physical model data of step 205, using materials with selected properties.

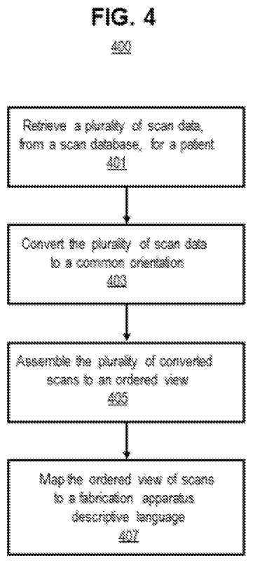

FIG. 4 illustrates a process 400 that may be used to design a patient-specific replacement body part. Process 400 may begin at step 401, at which a plurality of scan data, from scan database 105, may be retrieved for a patient. Next, process 400 proceeds to step 403 at which server 103 may convert the plurality of scan data to a common orientation. For example, the scan data may be converted to a predetermined angular view, resolution, size, and so forth. Next, process 400 proceeds to step 405 at which server 103 may assemble the plurality of converted scans to an ordered view. For example, the converted scans may be reordered to a sequential set of slices from top to bottom of a knee joint. Next, process 400 proceeds to step 407 at which server 103 may map the ordered view of scans to a fabrication apparatus descriptive language.

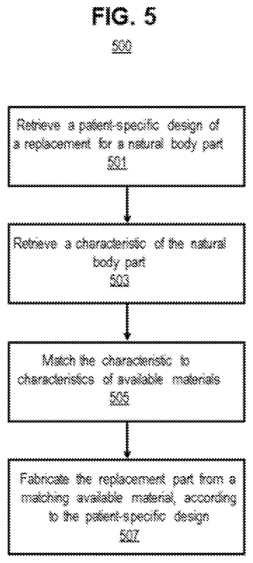

FIG. 5 illustrates a process 500 that may be used to design a patient-specific replacement body part. Process 500 may begin at step 501, at which a patient-specific design of a replacement for a natural body part may be retrieved. The design may be retrieved from a non-volatile memory such as database 105, and the design may be created by an application such as application program 311, using data obtained from system 100.

Next, control of process 500 progresses to step 503, at which a characteristic of the natural body part may be retrieved. For example, a texture of a patient's natural skin may be retrieved. Additional characteristics of the natural body part may be retrieved, such as color or softness. Specific characteristics that are retrieved may be dependent upon, and appropriate to, the natural body part. As a counterexample, hardness would be unlikely to be appropriate for natural skin.

Next, control of process 500 progresses to step 505, at which the retrieved characteristic(s) of the natural body part is compared to, and matched to, the corresponding characteristics of materials that might be available to fabricate a patient-specific replacement body part. The required closeness of a matching material selected from the available materials may be controlled by a designer using the system, e.g., by setting a tolerance level for the maximum devia-tion between the characteristic of the natural body part and the characteristic of the available material.

Next, control of process 500 progresses to step 507, at which the patient-specific replacement body part may be fabricated from the selected matching material, according to the patient-specific design.

### Mechanical Fastening Designs

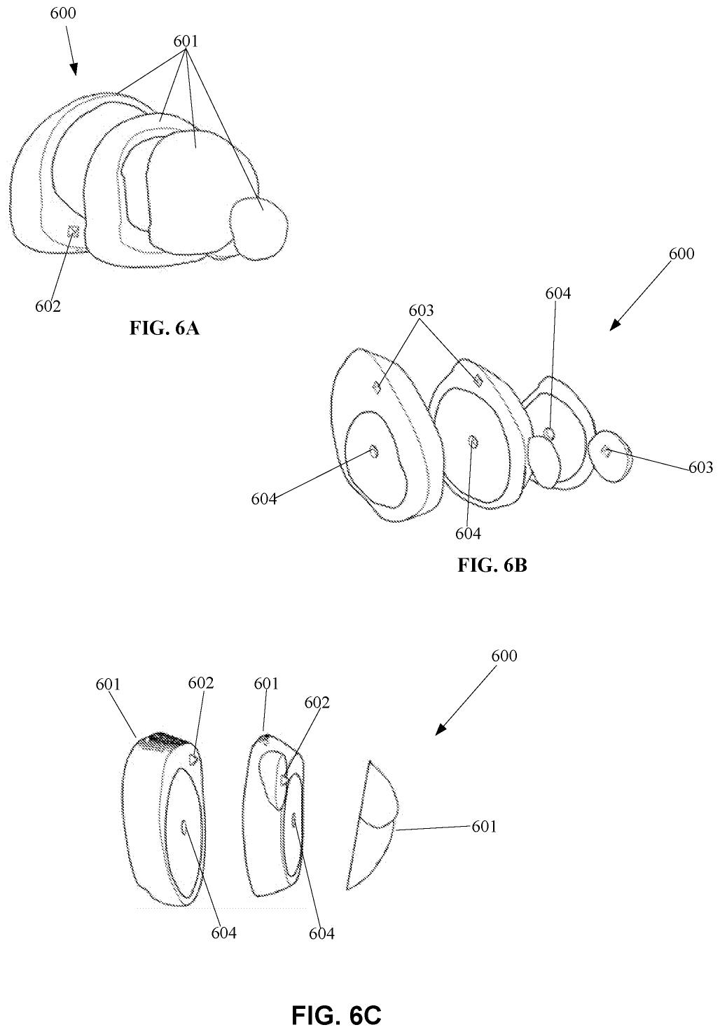

Now that a physical model can be obtained of a patient specific body part, means of displaying such a three-dimensional object in manners that allow for display of internal structure in cross section are illustrated in reference to FIG. 6A. Item 600 may represent a portion of a particular organ of a patient that may have been produced from a model generated by a medical imaging study. The model may be produced in multiple sections 601 which cut from the edge to the core of the model. In some examples, the individual sections may be produced in their discrete form. In other examples, the model may be produced as a whole and subsequently segmented by a sectioning technique such as mechanical cutting, laser cutting, water jet cutting or other means which can generate a set of sectioned pieces which may fit together snuggly.

Into each of the sectioned pieces a protrusion 602 may be placed or affixed upon a determined location upon the sectional piece. In some examples, where the individual sections are formed in discrete form, the process such as 3-D printing may also produce a protrusion or may produce a feature such as a recess into which a standard protrusion component may be placed and/or affixed. On a corresponding section piece which sits adjacent to the surface of the first piece which has the protrusion 602, a component may be located which receives the standard protrusion and locks it into orientation.

Referring now to FIG. 6B, a corresponding recess 603 is illustrated. The illustrated protrusion 602 is a skewed-pyramid shaped feature which protrudes from one surface and aligns with a reciprocal engraving in its adjacent mate. The protrusions may assume various shapes such as spherical, rectangular, triangular, n-hedron protrusions. Other interlocking-type features may include press-fit features (with or without additional materials, such as rubberized over-molded feature). Various other more complicated shapes may include features that are toothed, checkerboarded, slotted, or dovetailed in some examples. Other physical means of alignment and orientation to constrict in plane movements and rotations may include placement of magnets, placement of physical hoops and hooks and similar such alignment features.

The section pieces may include a centering device 604. In some examples, the centering device 604 may be formed initially of a cavity into which a magnet is placed. Each sectioned piece may include a centering device 604. There may be other interlocking devices other than magnets such as snaps, or Velcro® strips which may also be utilized.

In some examples, the model may have complicated shapes such as secondary lobes 603 which may be parts of the organ or abnormalities. Such a lobe 603 is illustrated with an exemplary low amount of overlap with the general structure of the organ. In such cases, the lobes 603 may also include protrusions (hidden) as well as recesses 603 for their own structure.

Referring now to FIG. 6C, another view of the exemplary model 600 is presented. In this alternative view, the relative orientation of the centering devices 604 and the protrusion 602 alignment devices is illustrated from a different angle.

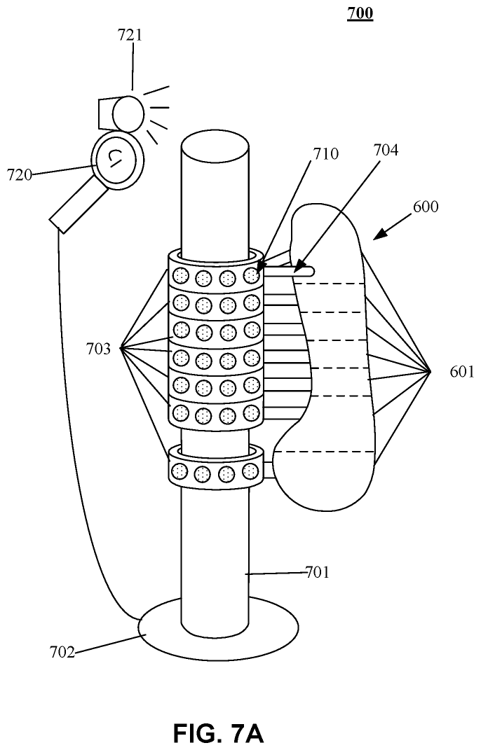

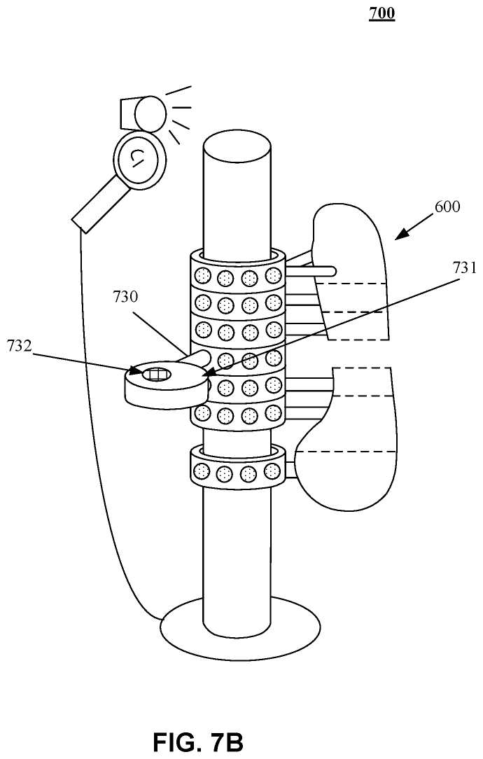

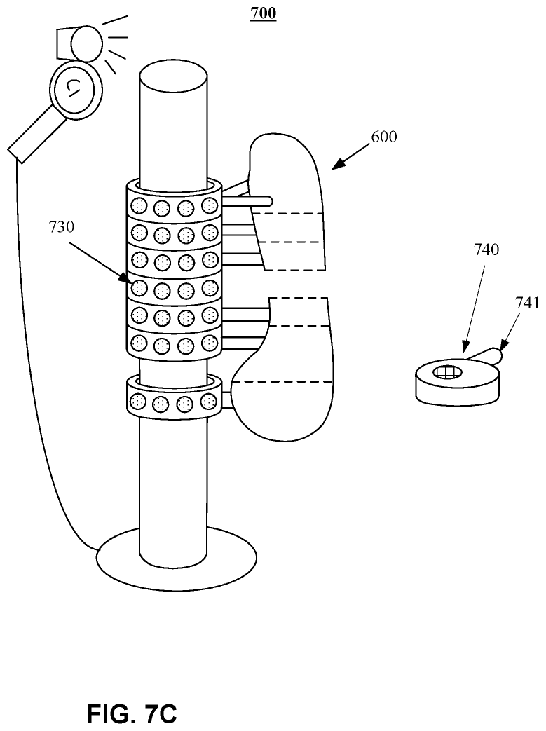

Referring to FIG. 7A a second exemplary type of fastened and sectioned three-dimensional model 700 expands upon the example illustrated in FIG. 6A by suspending a length of mating contacts along an axle. In some examples, the model 600 may comprise multiple sections 601. In the example of FIG. 7A, an axle 701 is supported upon a stand to ultimately support the model. The axle provides vertical location and support for the individual component sections. In an example, fastening pieces 703 may be positioned in the vertical dimension. The individual fastening pieces 703 may have the ability to rotate and may be formed with such features as bearings or other means to allow rotation while holding a vertical dimension fixed.

In some examples, the fastening pieces may be covered with a plurality of fastening devices 710 which may be used to hold an individual section piece 601 in place. Each of the section pieces 601 may have at least one and sometimes two or more fastening arms 704 which can attach to the fastening device and be affixed to an individual sectional piece 601.

In some examples, the models may have a base 702 which is stable and may have articulating abilities. As well the model may have built-in tools such as a magnifying glass 720 and light bulb 721. In some examples, various covers and decorations may be utilized to complete the appearance of the model structure.

A sectional piece 601 may be inspected by a user by rotating the section around the supporting axle. Referring to FIG. 7B, an example of a rotated section 731 is illustrated. The fastening device 730 rotates along with the supporting piece. The rotated section piece 731 exposes the surfaces of the section and illustrates internal structure 732.

A section piece 601 may also be removed from the model and inspected as a separated piece. Referring to FIG. 7C, an example of an unfastened piece 740 is illustrated separated from the supporting structure. The various fastening devices may allow for the piece to be easily re-attached and rotated into place within the model body. The fastening devices may include numerous types of devices. In a first example, the device may simply include a combination of magnets, one on the fastening device 730 and one on the corresponding supporting arm 741. Other such attachment devices may be used.

In a second example, a physical interlocking structure such as a dovetail joint may be cut into the fastening device and the support arm. The model section supporting arm may then be physically inserted into the joint and fastened thereby. In similar examples, the dovetail joint may be proximate to a ledge which may provide additional supporting strength to the fastening structure.

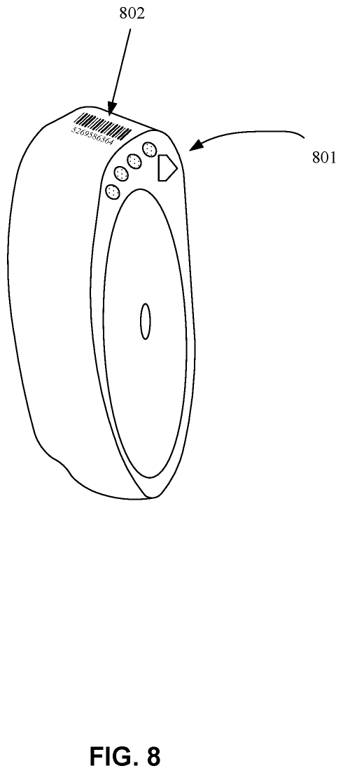

Referring now to FIG. 8, in another aspect, disparate pieces to be included in a model may include one or more of a) coupling mechanisms within the body of the respective pieces 801 and b) coupling mechanisms affixed to a surface of the respective. For example, it is within the scope of this invention to 3-D print adjacent and/or proximate pieces that may be assembled into a model, wherein the pieces include one or more magnetic areas near or one a surface to be coupled to an adjoining piece. Coupling mechanisms may be 3-D printed into a model piece or places within the model piece via one or both of manual placement of a coupling mechanism and placement via an automation, such as a robot. In some preferred embodiments, a pattern 801 of joining mechanisms is included. The pattern may be linear and essentially include a binary number, or a two-dimensional or three-dimensional patterns. A pattern may also include a machine-recognizable pattern 802, such as a hash. A machine recognizable pattern 802 allows the pattern to be meaningful in multiple ways, including acting as a coupling device and acting as an identifier. The identifier, for example, may include a unique pattern of coupling material that may be used to identify a particular piece of a model. The identification may be associated, for example with a particular patient. Accordingly, pieces of a first patient may be easily segregated from pieces of a second patient.

In some embodiments, the adjoining pieces are adjacent; in other embodiment two adjoining pieces may interlock or surround another piece that does not include a coupling mechanism. For example, a model of a kidney that includes kidney stone may include multiple pieces representing various portions of the kidney. The pieces representing kidney portions may include magnetic areas within the body of the pieces. Other piece(s) may represent the stone and not include magnetic coupling mechanisms but be contained within the pieces representing the kidney. In this manner, pieces may be assembled into a model with some pieces that are coupled via coupling mechanisms and others that are physically contained by and/or interlocked with other pieces.

In still another aspect, a complex model, such as a model of a human organ combined with biological features, such as one or more components of: the circulatory system (arteries and veins) and the nervous system, may include coupling mechanisms that are patterned for correct assembly. The patterns for correct assembly facilitate ease of reassembly if the model is partially or completely disassembled. Patterned coupling mechanisms may include, for example discrete areas of magnetic polarization of a first piece that is complimented by a mating pattern of opposite magnetic polarization on a second piece. When the first piece and second piece are placed proximate to each other, the complimented magnetic forces will cause a coupling. This may be contrasted with placing two pieces of non-complimented areas of magnetic polarization proximate to each other, wherein some areas may attract, and some areas may repel each other and the totality of attracting and opposing forces will not result in a proper coupling. In this manner, mistakes in assembly of a model may be avoided.

Relatedly, a database of patterns of magnetic forces may be maintained such that a scan of a pattern of magnetic areas present in a particular piece may be compared to the database and used to identify a model to which the piece is associated. In this manner, for example, and continuing the example of the kidney with stones, a piece from a model of a first kidney can be distinguished from a piece of a model of a second kidney.

In still a further aspect, a human-ascertainable indicator may be included on various pieces of a model that indicate mating surfaces. For example, a human-ascertainable indicator may include a small colored area, an alphanumeric number, a pattern, or other identifier. The human ascertainable indicator may also be a visual representation of a pattern of magnetic areas.

It is also within the scope of this invention for a user to indicate one or both of an artifact and an area of interest included on a scan. Automated model dissection may be used to generate one or more potential models wherein the potential models include different patterns of pieces to be included in a three-dimensional model. For example, a model of the kidney may include essentially planar dissections or may be dissected into irregular shapes that allow for better access to a specified aspect of the model. By way of non-limiting example, a user may indicate that a kidney stone is an area of interest (or one or more of: a pancreatic duct, or a blockage in an artery, a blood clot, a tumor, a fracture in a bone, a deteriorated spinal disc, a tear in a retina, area inflammation or other physiological aspect). Indication may be made via an interactive user interface on an automated device with a processor. The automated systems may execute routines that present one or more viable tangible model options for models that may be generated, such as via 3D printing. Each tangible model option may include different dissections. A user may make a selection of one or more virtual models to be manifested as physical tangible models.

In some embodiments, the automated systems may suggest coupling mechanisms and/or support structures that may be included in the one or more model options. A user may select a preferred coupling mechanism to be included in the respective model(s) chosen. Preferred coupling mechanisms include areas of 3D printed magnetic material.

As examples of physical models which may be presented in a manner that allows for easy presentation of cross-sectional structure as well as the overall structure examples of medical body parts and other medical related models have been discussed. It may be apparent that the techniques and apparatus as have been described may have general applicability to other types of physical models, particularly those whose internal structure is elucidated by a measurement means which can sense parametric variation across the three-dimensional structure. Accordingly, it will be understood that various modifications may be made without departing from the spirit and scope of the claimed disclosure.

### CONCLUSION

A number of embodiments of the present disclosure have been described. While this specification contains many specific implementation details, there should not be construed as limitations on the scope of any disclosures or of what may be claimed, but rather as descriptions of features specific to particular embodiments of the present disclosure. While embodiments of the present disclosure are described herein by way of example using several illustrative drawings, those skilled in the art will recognize the present disclosure is not limited to the embodiments or drawings described. It should be understood the drawings and the detailed description thereto are not intended to limit the present disclosure to the form disclosed, but to the contrary, the present disclosure is to cover all modification, equivalents and alternatives falling within the spirit and scope of embodiments of the present disclosure as defined by the appended claims.

The headings used herein are for organizational purposes only and are not meant to be used to limit the scope of the description or the claims. As used throughout this application, the word “may” is used in a permissive sense (i.e., meaning having the potential to), rather than the mandatory sense (i.e., meaning must). Similarly, the words “include”, “including”, and “includes” mean including but not limited to. To facilitate understanding, like reference numerals have been used, where possible, to designate like elements common to the figures.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

The term “a” or “an” entity refers to one or more of that entity. As such, the terms “a” (or “an”), “one or more” and “at least one” can be used interchangeably herein. It is also to be noted the terms “comprising”, “including”, and “having” can be used interchangeably.

Certain features that are described in this specification in the context of separate embodiments can also be implemented in combination in a single embodiment. Conversely, various features that are described in the context of a single embodiment can also be implemented in combination in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while method steps may be depicted in the drawings in a particular order, this should not be understood as requiring that such operations be performed in the particular order shown or in a sequential order, or that all illustrated operations be performed, to achieve desirable results.

Certain features that are described in this specification in the context of separate embodiments can also be implemented in combination in a single embodiment. Conversely, various features that are described in the context of a single embodiment can also be implemented in combination in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Moreover, the separation of various system components in the embodiments described above should not be understood as requiring such separation in all embodiments, and it should be understood that the described program components and systems can generally be integrated together in a single software product or packaged into multiple software products.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order show, or sequential order, to achieve desirable results. In certain implementations, multitasking and parallel processing may be advantageous. Nevertheless, it will be understood that various modifications may be made without departing from the spirit and scope of the claimed disclosure.

What is claimed is:

1. A system to support a sectioned model of a patient-specific body part model for a natural body part, comprising:
a server having a processor and a memory, the memory comprising stored instructions to be executed by the processor, the server coupled to a user terminal and to a communication network;
a fabrication apparatus coupled to the server through the communication network, the fabrication apparatus fabricating the patient-specific body part model,
wherein the stored instructions, when executed by the server, perform the steps of:
retrieving a patient-specific design of a natural body part, from a calculation performed upon a medical imaging database comprising data values of medical imaging studies of the patient;
fabricating the patient-specific body part model from the selected matching material, according to the patient-specific design;
wherein the fabricating of the patient-specific body part is performed in a multitude of fabricating steps and wherein each of the multitude of fabricating steps produces a discrete section of the patient-specific body part model.

2. The system to support a sectioned model of a patient-specific body part of claim 1 wherein each of the discrete sections of the patient specific body part model comprises a fastening device, wherein the fastening device fastens at least a first discrete section to at least a second discrete section.

3. The system to support a sectioned model of a patient-specific body part of claim 2 wherein the fastening device comprises a magnet.

4. The system to support a sectioned model of a patient-specific body part of claim 3 wherein the magnet is printed with a printer capable of printing three-dimensional objects (i.e., a 3-D printer).

5. The system to support a sectioned model of a patient-specific body part of claim 2 wherein the fastening device comprises a hook-and-loop fastener device.

6. The system to support a sectioned model of a patient-specific body part of claim 2 wherein the fastening device comprises a physical interlocking structure.

7. The system to support a sectioned model of a patient-specific body part of claim 1 wherein each of the discrete sections of the patient specific body part model comprises at least a first orienting device.

8. The system to support a sectioned model of a patient-specific body part of claim 7 wherein the first orienting device comprises a recessed shape on a first discrete section and a corresponding protruding shape on a second discrete section.

9. The system to support a sectioned model of a patient-specific body part of claim 8 further comprising a second orienting device, wherein the second orienting device comprises a recessed shape on the first discrete section and a corresponding protruding shape on the second discrete section.

10. The system to support a sectioned model of a patient-specific body part of claim 1 wherein each of the discrete sections of the patient specific body part model comprises a fastening device, wherein the fastening device fastens at least a first discrete section to at least a second discrete section.

11. The system to support a sectioned model of a patient-specific body part of claim 10 wherein each of the discrete sections of the patient specific body part model further comprises an orienting device.

12. A system to support a sectioned model of a patient-specific body part model for a natural body part, comprising:
a server having a processor and a memory, the memory storing instructions to be executed by the processor, the server coupled to a user terminal and to a communication network;
a fabrication apparatus coupled to the server through the communication network, the fabrication apparatus fabricating the patient-specific body part model,
wherein the stored instructions, when executed by the server, perform the steps of:
retrieving a patient-specific design of a natural body part, from a calculation performed upon a medical imaging database comprising data values of medical imaging studies of the patient;
fabricating the patient-specific body part model from the selected matching material, according to the patient-specific design; and
wherein the fabricating of the patient-specific body part is performed in a multitude of fabricating steps and wherein each of the multitude of fabricating steps produces a discrete section of the patient specific body part model;
a support axle; and
a plurality of fastening pieces, wherein the fastening pieces can rotate around the support axle while being affixed thereupon.

13. The system to support a sectioned model of a patient-specific body part model for a natural body part of claim 12 wherein each of the discrete sections of the patient-specific body part model comprises a fastening device, wherein the fastening device fastens at least a first discrete section to at least a second discrete section.

14. The system to support a sectioned model of a patient-specific body part model for a natural body part of claim 13 wherein the fastening device comprises a magnet.

15. The system to support a sectioned model of a patient-specific body part model for a natural body part of claim 13 wherein the magnet is printed with a printer capable of printing three-dimensional objects (i.e., a 3-D printer).

16. The system to support a sectioned model of a patient-specific body part model for a natural body part of claim 13 wherein each of the discrete sections of the patient-specific body part model further comprises at least a first orienting device.

17. A method for presenting a model comprising:
performing a medical imaging study upon a patient;
creating a digital model of a three-dimensional representation of a body part of the patient;
fabricating a physical model utilizing the digital model as input to a processing tool, wherein the fabricating of the physical model is performed in a multitude of fabricating steps and wherein each of the multitude of fabricating steps produces a discrete section of the patient-specific body part mode;
affixing a fastening device upon each of the multitude of discrete sections as a centering device;
fabricating alignment devices upon each of the plurality of sections; and
assembling the sections into an aligned composite of layers.

18. The method for presenting a model of claim 17 wherein the fastening device is a magnet.

19. The method for presenting a model of claim 17 wherein the alignment devices upon each of the plurality of sections are fabricated in the same step as the discrete sections.

20. The method for presenting a model of claim 19, wherein the multitude of fabrication steps are printed three dimensionally.
